(19) United States
(12) Patent Application Publication
(10) Pub. No.: US 2020/0038235 A1
(43) Pub. Date: Feb. 6, 2020

(54) Personal Extremity Warming Device

(71) Applicant: Kevin Lutsky, Linwood, NJ (US)

(72) Inventor: Kevin Lutsky, Linwood, NJ (US)

(21) Appl. No.: 16/454,142

(22) Filed: Jun. 27, 2019

Related U.S. Application Data
(60) Provisional application No. 62/692,659, filed on Jun. 30, 2018.

(51) Int. Cl. A61F 7/03

(52) U.S. Cl. CPC A61F 7/034; A61F 2007/0035

(57) ABSTRACT

A personal extremity warming apparatus having a band, a pocket attached to the band, and an opening adapted to receive a heating element, the pocket having a front panel and rear panel the front panel adapted to attach to said rear panel along all but one of a plurality of front and rear panel sides. In an embodiment of the invention, the personal extremity warming apparatus has a band, a rear panel fixedly attached to the band along a first long side, a first short side, and a second short side, and the rear panel further having a second long side of the rear panel forming an opening adapted for receiving a heating element.

### FIELD OF THE INVENTION

The present invention generally relates to the field of clothing, in particular, devices for warming hands and feet.

### BACKGROUND INFORMATION

Anatomical extremities, such as hands and feet, are powerful thermoregulators in the body forming thermal insulators in the cold and heat radiators in hot environments. Hands and feet have keen thermal sensors that provide feedback to the brain that affects thermoregulatory behavior. In cold environmental conditions, the extremities are particularly susceptible to damage.

When exposed to cold environments, the body lowers blood supply to the extremities by constricting blood vessels in order to preserve body heat in and protect the core. This vasoconstriction along with the high surface area-to-volume ratio, skin temperatures in fingers and toes tend to quickly decrease to a level approaching the cold ambient environment. Without protective clothing or other cold-mitigating devices hands and feet, especially fingers and toes, may experience tactile sensitivity including numbness, reduced manual dexterity, loss of motor function, necrosis, immersion foot, frostbite, tissue crystallization, or loss of limb. Therefore, in occupational and recreational activities occurring in cold environs it is imperative to counter the ambient temperature by protecting anatomical extremities.

While gloves and thermal socks provide an insulating layer of protection between anatomical extremities and cold ambient temperatures, reducing the rate of heat loss through the hands and feet, such gloves and thermal socks merely mitigate the time taken for non-freezing cold injuries to set in due to continued cold exposure. Many devices have been developed and used over the years to provide direct and portable heating to the anatomical extremities. Such devices include hand-held warmers utilizing electricity, combustible fuels, or chemical compounds reacting exothermically. Such hand-held warmers are usually inefficiently positioned in the palm of the hand, against the back of the hand, or tucked in a jacket pocket. Because of where hand-held warmers are placed, such hand-held warmers are inefficient and can impact fine and gross motor function. Specifically, if the hand warmer is held against the palm, and hands and fingers must hold the hand warmer in place and cannot be used for other activities. Where the hand warmer is placed against the back of the hand, venous blood travelling away from the fingers is warmed. Keeping the hand warmer in a jacket pocket means that the hands must stay in the jacket pocket rendering them useless or non-continuous warming when the hands are taken out of the jacket pocket. Similar issues are present in warming devices for feet.

Therefore, a warming device that warms arterial blood flowing towards anatomical extremities and encourages vasodilation bringing more blood to fingers and toes is preferential.

### BRIEF DESCRIPTION OF THE DRAWINGS

The present disclosure is best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion

FIG. 1 depicts a personal extremity warming apparatus consistent with certain embodiments of the present disclosure.

FIG. 2 depicts a personal extremity warming apparatus consistent with certain embodiments of the present disclosure.

FIG. 3 depicts a method for using a personal extremity warming apparatus consistent with certain embodiments of the present disclosure.

### DETAILED DESCRIPTION OF THE PREFERRED EMBODIMENTS

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

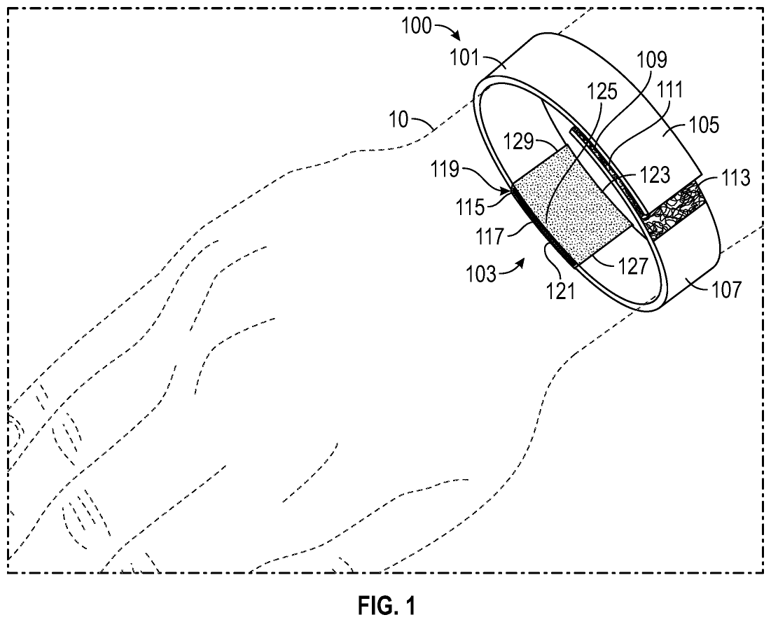

FIG. 1 depicts personal extremity warming apparatus positioned around wrist 10 of a user to provide warmth to hands and fingers. As shown in FIG. 1, personal extremity warming apparatus, generally designated by reference numeral 100, has band 101 and pocket 103. Band 101 may be an elastic sleeve stretching to adjust to the size of user's wrist 10 or ankle. In certain embodiments, band 101 may be integrated into personal clothing. In certain embodiments, band 101 may be a portion of a shirt sleeve, a portion of a mitten, a portion of a glove, a portion of a sock, and a portion of a pant leg. In certain embodiments, band 101 may be adapted to fixedly secure around a portion of a human body such that band 101 does not shift in response to movement of human body. In some embodiments band 101 may be an adjustable strap. In some embodiments, band 101 may be fixed length. In some embodiments, band 101 may be elongated continuous material having first end 105 and second end 107. In certain embodiments, first end 105 may be secured to second end 107. In certain embodiments fastener 109 may secure first end 105 to second end 107. Fastener 109 may include securing side 111 and receiving side 113. Examples of fastener 109 may include hook-and-loop fasteners such as Velcro, slide-release fasteners, clips, buckles, clasps, and ring fasteners. In certain embodiments, securing side 111 may be part of first end 105 and receiving side 113 may be part of second end 107.

Pocket 103 is adapted to receive and remove heating element 115 at opening 117. In certain embodiments, pocket 103 houses non-removable heating element 115. In some embodiments, pocket 103 is affixed to band 101. As shown in FIG. 1, pocket 103 has rear panel 117, front panel 119, and opening 121. Rear panel 117 and front panel 119 are similar size and shape. Pocket 103 may be made of any breathable material to allow air flow to heating element 115 including fabric or mesh. In certain embodiments, pocket 103 is integrated into a portion of personal clothing. In certain embodiments, pocket 103 is integrated into a portion of a shirt sleeve, a portion of a pant leg, a portion of a mitten, a portion of a glove, and a portion of a sock. In certain embodiments, front panel 119 is made of heat reflecting material to efficiently direct heat from heating element 115 towards wrist. Pocket 103 may have any shape adapted to fit heating element 115. As depicted in FIG. 1, pocket 103 is a rectangular shape having long sides 123, 125 and short sides 127, 129. Rear panel 117 and front panel 119 are affixed at long sides 123, 125 and short sides 127, 129. In certain embodiments, pocket 103 has a single panel of continuous folded material fixedly attached at short sides 127, 129 or long sides 123, 125. In some embodiments, rear panel 117 and front panel 119 may be affixed via zipper, snaps, or hook-and-loop fasteners. In certain embodiments, rear panel 117 and front panel 119 may be fixedly attached along at least one of long sides 123, 125 and short sides 127, 129. Opening 121 may form along at least one of the non-fixedly attached long sides 123, 125 and short sides 127, 129. In certain embodiments, opening 121 may be on front panel 119.

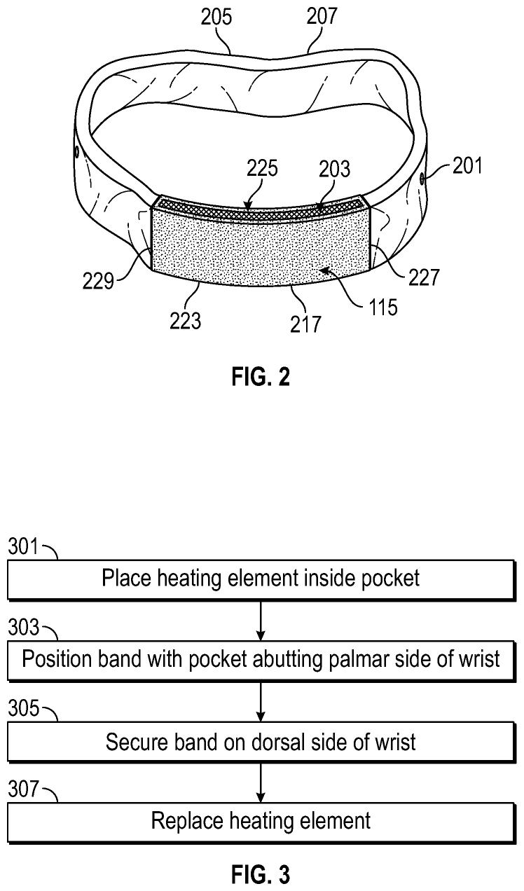

FIG. 2 depicts an alternative embodiment of personal extremity warming apparatus. As shown in FIG. 2, band 201 is an elastic sleeve with first end 205 fixedly attached to second end 207. In certain embodiments, elastic sleeve of band 201 is a tubular formed by folding a continuous rectangular piece of an elastic material having elongated horizontal edges and short vertical edges and stitching elongated horizontal edges of the material together. Single rectangular panel 217 having long sides 223, 225 and short sides 227, 229 is fixedly attached to band 201 at short sides 227, 229 and long side 223 forming pocket 203. Opening 221 forms along unaffixed long side 225. In certain embodiments, long sides 223, 225 and short sides 227, 229 are all affixed to band 201 and opening 221 is a slit along a front portion of panel 217 for the insertion and removal of heating element 115. In certain embodiments, opening 221 may be closed via zipper, snaps, or hook-and-loop fasteners. In certain embodiments, pocket 203 is a centrally located part of band 201, pocket 203 has stitching along shorts sides 223, 225 to secure heating element 115 in place, and opening is a slit along front portion of pocket 203. This structure allows band 201 and pocket 203 to be formed of one continuous material that can stretch to secure around different sized wrists or ankles yet secure removable heating element 115 in place along wrist.

FIG. 3 is a flow chart of a method for using personal extremity warming apparatus 100 in accordance with some embodiments. Heating element 115 is placed inside pocket 103 of the personal extremity warming apparatus 100. In certain embodiments, heating element 115 is inserted into pocket 103 through opening 121. In some embodiments, rear panel 117 is detached from front panel 119 and heating element 115 is placed on rear panel 117. Front panel 119 is placed over heating element 115 and affixed to rear panel 117. In certain embodiments, heating element 115 is placed in pocket 103 before placing band 101 around wrist or ankle of user. In some embodiments, heating element 115 may be inserted into pocket 103 after band 101 is positioned on body of user.

Band 101 is placed around anatomical appendage, for example, around wrist 10 of a user. Band 101 is initially positioned such that first end 105 and second end 107 will attach on dorsal or back side of wrist 10 of user and pocket 103 is positioned against palmar or inner side of wrist 10 of user. In certain embodiments, band 101 is secured by engaging securing side 111 and receiving side 113 thereby attaching first end 105 to second end 107 of band 101. First end 105 and second end 107 may overlap such that, for example, hook-and-loop material on securing side 111 may engage hook-and-loop material on receiving side 113. In some embodiments, band 101 is an elastic sleeve and hand is threaded through band 101 until band 101 reaches wrist 10. In such an embodiment, the elastic properties of sleeved band 101 will cause band 101 to stretch to circumference of hand and retract to circumference of wrist 10 securing in place against wrist 10 of user.

When band 101 is in place, around wrist 10 of user, personal extremity warming apparatus 100 is positioned such that pocket 103 abuts palmar-side or inner-side of wrist 10 of user. Heating element 115 radiates heat against wrist 10 of user warming arterial blood flowing through wrist 10 and traveling to hands and fingers of user also resulting in dilation of blood vessels thereby bringing additional warmed blood to tips of fingers. Once heating element 115 no longer provides sufficient heat to maintain warmth, heating element 115 may be removed and replaced. Removing heating element 115 may necessitate first end 105 is separated from second end 107 of band 101 such that band 101 is opened. In certain embodiments, heating element 115 may be removed and replaced by accessing heating element 115 though opening 121.

While the principles of the invention have been described herein, it is to be understood by those skilled in the art that this description is made only by way of example and not as a limitation as to the scope of the invention. Further embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. A personal extremity warming apparatus, comprising:
a band;
a pocket, said pocket attached to said band, said pocket comprising a front panel and rear panel, said front panel having a plurality of sides, said rear panel having a plurality of sides, said front panel adapted to attach to said rear panel along all but one of said plurality of front panel sides and said plurality of rear panel sides, said rear panel adapted to attached to said front panel along all but one of said plurality of rear panel sides and said plurality of front panel sides; and
and opening adapted to receive a heating element.

2. The personal extremity warming apparatus of claim 1 wherein said front panel is adapted to attach to said rear panel along all of said plurality of front panel sides and said plurality of rear panel sides, said rear panel adapted to attach to said front panel along all of said plurality of rear panel sides and said plurality of front panel sides.

3. The personal extremity warming apparatus of claim 1 wherein said front panel is fixedly attached to said rear panel along all of said plurality of front panel sides and said plurality of rear panel sides and said rear panel is fixedly attached to said front panel along all of said plurality of rear panel sides and said plurality of front panel sides.

4. The personal extremity warming apparatus of claim 1 wherein said front panel and rear panel comprise a continuous material.

5. The personal extremity warming apparatus of claim 1 wherein said band comprises an elastic sleeve.

6. The personal extremity warming apparatus of claim 1 wherein said band comprises a strap, said strap having a first end and second end, said first end is attached to said second end.

7. The personal extremity warming apparatus of claim 1 wherein said band comprises a strap and a fastener, said strap having a first end and second end, said first end is secured to said second end by said fastener.

8. The personal extremity warming apparatus of claim 1 wherein said opening comprises a slit on said front panel.

9. A personal extremity warming apparatus, comprising:
a band;
a rear panel, said rear panel comprising a first long side, said first long side fixedly attached to said band, said rear panel further comprising a first short side and a second short side, said first short side and second short side fixedly attached to said band; and
an opening adapted to receive a heating element.

10. The personal extremity warming apparatus of claim 9 wherein said opening comprises a slit on surface of said rear panel.

11. The personal extremity warming apparatus of claim 9 wherein said rear panel further comprises a second long side, said second long side forming said opening.

12. The personal extremity warming apparatus of claim 9 wherein said band comprises an elastic sleeve.

13. The personal extremity warming apparatus of claim 9 wherein said band comprises a strap, said strap having a first end and second end, said first end is attached to said second end.

14. The personal extremity warming apparatus of claim 9 wherein said band comprises a strap and a fastener, said strap having a first end and second end, said first end is secured to said second end by said fastener.
